(19) United States
(12) Patent Application Publication
(10) Pub. No.: US 2008/0097356 A1
(43) Pub. Date: Apr. 24, 2008

(54) Personal hygiene wipe

(76) Inventor: James A. Donovan, Odessa, FL (US)

Correspondence Address: LAW OFFICES OF JOHN S. MUNDAY, P.O.BOX 423, Isanti, MN, 55040, US

(21) Appl. No.: 11/584,145

(22) Filed: Oct. 20, 2006

(51) Int. Cl. A61F 7/00

(52) U.S. Cl. 604/291

(57) ABSTRACT

A personal hygiene wipe that includes a contained and controlled exothermic chemical reaction to provide warmth for the wipe. The fabric of the wipe, such as a rayon and polyester fabric also includes a first or chemical outer pouch, a second or water containing pouch inside the first pouch, such that the first pouch is sealed with a vacuum so that upon application of pressure to the wipe, the vacuum increases the spread of the water into contact with the chemicals to initiate the exothermic reaction.

### FIELD OF THE INVENTION

This invention relates to a personal hygiene wipe or cloth used to cleanse various parts of the body. More particularly, the invention relates to a personal hygiene wipe or cloth that is temperature controlled to give increased comfort and utility.

### BACKGROUND OF THE INVENTION

Personal hygiene wipes are often used by persons when they are away from their home and do not have access to a shower or bath. They are also used when the part of the person that is to be cleaned is small, and a shower or bath consumes too much time.

In order to be effective, however, personal hygiene wipes need to be warmed or heating in order to more effectively clean the hands or other parts of the user's anatomy. At the present time, warm wipes are only attainable by the use of an external source of hot water, or by inserting the wipes into a microwave or other heating device. This presents a danger as the degree of heating may vary, and it is possible to have excessive heat applied to the skin.

It has been suggested that some form of exothermic reaction could be used to generate heat in these products. One such suggestion is to employ a supercooled liquid in a container that can be disturbed by the user at the appropriate time, thus causing an exothermic reaction as the liquid crystallizes. However, because personal hygiene wipes are carried in various purses, brief cases, and other containers, and because they may be subject to forces that are not anticipated, early crystallization of the supercooled liquid causes the wipes to be hot at a time when that is not needed.

Another major drawback of the use of an exothermic reaction to generate heat upon demand is that the various components have to be kept totally separated from each other until they are combined, and when combined need to react quickly and over a reasonable surface area. If the reaction only takes place at one location, excessive heat will be generated. If the reaction components are spread out, there has not been any way to combine them from the dispersed locations to generate uniform exothermic reaction.

It would be a great advantage if a way of heating personal hygiene wipes could be developed that have a controlled release of heat that is within acceptable safety limits.

Another advantage would be to provide a way of heating personal hygiene wipes that is controlled and requires a specific action by the user such that the action is not one experienced by the wipes when carried about prior to use.

Yet another advantage would be to provide a way to generate heat by an exothermic reaction over a personal hygiene wipe sized area quickly, without having to wait for an activation agent to make its way to all the reaction components.

Other advantages will appear hereinafter.

### SUMMARY OF THE INVENTION

It has now been discovered that the above and other objects of the present invention may be accomplished in the following manner. The unique aspect of this invention is that a controlled, dispersed exothermic reaction can be used to warm one or more personal hygiene wipes quickly and effectively.

In it's simplest form the invention comprises a package holding one or more personal hygiene wipes. Also inside the package is a system for generating warming heat over substantially all the surface of the wipe or wipes.

The system inside the package includes an actuatable heat source in contact with the hygiene wipe or wipes that has an inner pouch and an outer pouch. The inner pouch includes an actuation agent that is isolated until need, and some form of seal that can be broken or opened upon demand. A frangible seal is preferred. The outer pouch contains heat generating materials therein, where the heat generating materials are adapted to generate heat upon activation by the actuation agent. The outer pouch has an internal vacuum sufficient to pull the activation agent into said outer pouch upon opening said seal.

In the preferred embodiment, the heat generating materials are crystals that exotherm when contacted with a liquid actuating agent. The preferred actuation agent is water and the preferred heat generating material is a crystalline mixture of calcium oxide and citric acid. The weight of the heat generating crystalline material to the volume of actuation agent ranges from about 1:2 to about 1:1. By spreading the crystals throughout the outer pouch, heat generating reactions take place in a large number of locations, thus heating the wipe or wipes uniformly. A vacuum in the outer pouch increases the rate of flow of water to all regions of the pouch, causing uniform time of reaction.

The amount of vacuum can range from about 8 psi to about 13 psi, with about 11 psi being preferred.

### BRIEF DESCRIPTION OF THE DRAWINGS

For a more complete understanding of the invention, reference is hereby made to the drawings, in which:

FIG. 1 is a side elevational view of the preferred embodiment of the invention;

FIG. 2 is a perspective view of the device illustrating the removal of one element from the embodiment shown in FIG. 1;

FIG. 3 is a side elevational view of the inside components of the embodiment shown in FIG. 1; and

FIG. 4 is a sectional view taken along line 4-4 of FIG. 3.

### DETAILED DESCRIPTION OF THE PREFERRED EMBODIMENT

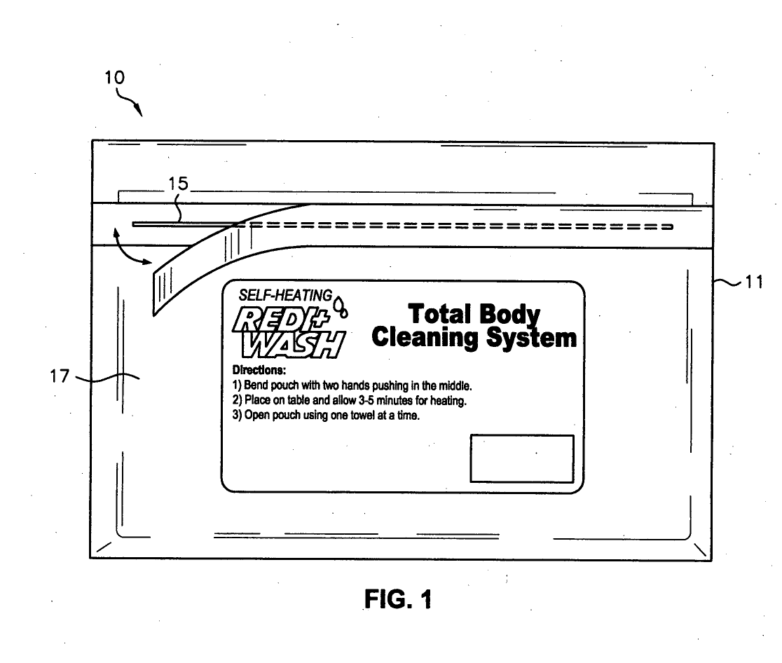

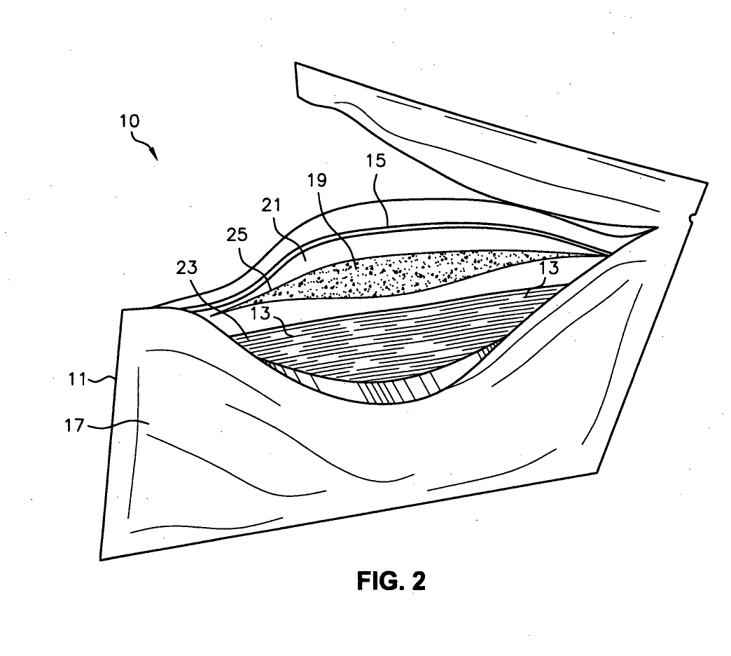

As shown in FIG. 1, the device of this invention, 10 generally, comprises a package 11 containing one or more personal hygiene wipes 13 of conventional design, shown in FIG. 2 after the zipper closure 15 has been opened. Preferred are those wipes made of spun lace fabric, such as those from rayon and polyester fibers.

Also enclosed in package 11 is an outer pouch 17 made of a fluid impervious material such as a plastic. Pouch 17 contains the heat generating materials 19 that exotherm when contacted by an actuating agent as described below. Heat generating materials 19 are preferably in crystal or granular form so that they can be spread throughout the inside of pouch 17, thus, when activated, providing exotherm heat over the whole wipes 13.

Preferred is an outer pouch 17 made from Aclar®, which is a polychlorotrifluoroethylene (PCTFE) material manufactured and sold by Honeywell International Inc. Aclar film is crystal clear, biochemically inert, chemical-resistant, nonflammable, and plasticizer- and stabilizer-free. Aclar laminates provide a wide range of gauges and thus barrier levels to allow flexibility in selecting the optimum barrier level for the chemical system chosen. Other similar pouch materials may be used as well. All that is required is that the material have a functional moisture and vapor barrier for the other components of the invention.

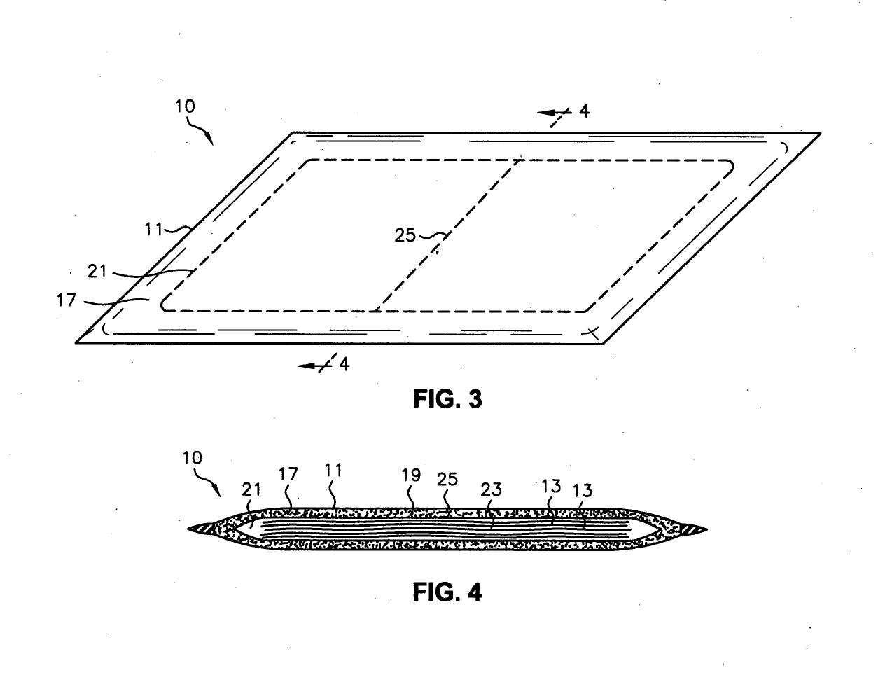

As seen in FIGS. 3 and 4, the package includes an inner pouch 21 positioned inside the outer pouch 17. The inner pouch 21 contains an activation agent 23 that combines with the heat generating materials 19 when the seal 25 in pouch 21 is opened. Preferably, seal 25 is a frangible seal that can be broken by flexing it.

Preferred is an inner pouch 21 made from a heat stamp foil such as.

Pouch 21 has a frangible seal 25 that is preferably broken at the appropriate time by flexing or bending the package 11 to cause the activating agent 23 to mix with the heat generating material 19 and exotherm at the many locations inside outer pouch 17.

In order to have an uniform and even production of heat from the exotherms, it is preferred that the inside of inner pouch 21 be at atmospheric pressure or 14.7 psi, and the inside of the outer pouch 17 be under vacuum. Preferred pressures in outside pouch 17 are from about 8 psi to about 13 psi, with 10 or 11 psi being preferred. It is necessary to have a pressure differential between the inside of both pouches to be sufficient to pull the activating agent 23 to the entire area where the heat generating material 19 has been placed. Too little or too great a pressure differential is not desired, for design and reliability reasons.

There are a number of combinations of heat generating materials and activating agents that are suitable for use in the present invention. The selection of specific components is to be based upon cost, compatibility, ease of control of the exotherm, and other factors.

The preferred activating material of this invention is water. This is plentiful and safe, and reacts with a number of materials to produce an exothermic reaction.

The preferred heat generating material is a crystal formed from several components that, when free from moisture, are stable for up to three to five years or more, and which react when moisture is present to generate heat. The preferred crystal is made from a crystalline mixture of calcium oxide and citric acid. The weight of the heat generating material to the volume of actuation agent ranges from about 1:2 to about 1:1, and preferably about 3:4. In the most preferred mixture of the heat generating material is a mixture of calcium oxide and citric acid with the further addition of a zeolite powder. Preferred is a ratio of calcium oxide to citric acid to powdered zeolite is from about 14 to 20 for calcium oxide, from about 1 to 2 for citric acid, and from about 7 to 10 for powdered zeolite. Most preferred is a ratio of calcium oxide to citric acid to powdered zeolite is 17:1.5:8.5 and the ratio of the solids to the volume of water is about 3:4.

More than 150 zeolite types have been synthesized and 48 naturally occurring zeolites are known. They are basically hydrated alumino-silicate minerals with an “open” structure that can accommodate a wide variety of positive ions, such as Na+, K+, Ca₂+, Mg₂+ and others. These positive ions are rather loosely held and can readily be exchanged for others in a contact solution. Some of the more common mineral zeolites are: analcime, chabazite, heulandite, natrolite, phillipsite, and stilbite. An example mineral formula is: Na₂Al₂Si₃O₁₀.16H₂O.

The heat generation material most preferred, using the above components includes a calcined calcium oxide. This material is available as a small article size, with a diameter less than about 0.2 mm, and as a particle of somewhere between 0.2 and 0.8 mm. Larger particles are ground and smaller ones sieved, and the calcium oxide is then calcined. It has been found to be effective to calcine for at least 60 to 120 minutes, and preferably about 90 minutes, at temperatures above 500° C., and most preferably at about 550° C. for that period of time. The calcined calcium oxide is, of course, desiccated to prevent any contamination by moisture. Laboratory grade citric acid and powdered zeolite are mixed with the calcium oxide in moisture free conditions, in an appropriate reaction ratio to provide the exothermic reaction upon contact by the activating agent water.

The heat generating mixture 19 is spread throughout the inside of outer pouch 17 and covers parts of inner pouch 21, obviously separated from the activating agent 23 inside pouch 21. The vacuum is then pulled inside pouch 17 to assist in dispersing the activating agent. This combination of pouches is placed with the desired quantity of personal hygiene wipes 15 inside package 11 and sealed for distribution and sale.

In a series of tests of the preferred embodiment as described above, 100% of the activations by bending the packages resulted in warm personal hygiene wipes. Then a similar set of packages were prepared, with the only change being no vacuum inside the outer pouch, only 30% of the wipes achieved the desired temperature,

While particular embodiments of the present invention have been illustrated and described, it is not intended to limit the invention to any specific embodiment. The description of the invention is not intended to limit the invention.

1. A heatable personal hygiene wipe device, comprising:
an outer package having a sealed opening for removing contents;
a plurality of hygiene wipes contained in said outer package;
an actuatable heat source in said outer package in contact with said plurality of hygiene wipes, said heat source including an inner pouch and an outer pouch;
said inner pouch having an actuation agent therein and a seal keeping said actuation agent in said inner pouch; and
said outer pouch having heat generating materials therein, said heat generating materials being adapted to generate heat upon activation by said actuation agent, said outer pouch having an internal vacuum sufficient to pull said activation agent into said outer pouch upon opening said seal.

2. The device of claim 1, wherein said sealed opening is re-sealable.

3. The device of claim 1, wherein said plurality of hygiene wipes is from about 3 to about 20.

4. The device of claim 1, wherein the amount of vacuum in said outer pouch is sufficient to give a pressure of from about 8 to 13 psi.

5. The device of claim 1, wherein said actuation agent is a liquid and said heat generating material is a solid adapted to react with said actuation agent to cause an exothermic reaction.

6. The device of claim 5, wherein said actuation agent is water and said heat generating material is a crystalline mixture of calcium oxide and citric acid, and the weight of the heat generating material to the volume of actuation agent ranges from about 1:2 to about 1:1.

7. The device of claim 6, wherein said calcium oxide is calcined prior to admixing with said citric acid.

8. The device of claim 7, which further includes powdered zeolite admixed therein.

9. The device of claim 8, wherein the ratio of calcium oxide to citric acid to powdered zeolite is from about 14 to 20 for calcium oxide, from about 1 to 2 for citric acid, and from about 7 to 10 for powdered zeolite.

10. The device of claim 9, wherein the ratio of calcium oxide to citric acid to powdered zeolite is 17:1.5:8.5 and the ratio of the solids to the volume of water is about 3:4.

11. A heatable personal hygiene wipe device, comprising:
outer package having sealed opening means for removing contents;
a plurality of hygiene wipes contained in said outer package;
an actuatable heat source means in said outer package in contact with said plurality of hygiene wipes for generating heat, said heat source means including an inner pouch and an outer pouch;
said inner pouch having an actuation means for actuating a chemical exotherm therein and a seal keeping said actuation means in said inner pouch; and
said outer pouch having heat generating materials means therein for generating heat upon contact by said actuation means, said outer pouch having an internal vacuum sufficient to pull said activation agent into said outer pouch upon opening said seal.

12. The device of claim 11, wherein said sealed opening means is re-sealable.

13. The device of claim 11, wherein said plurality of hygiene wipes is from about 3 to about 20.

14. The device of claim 11, wherein the amount of vacuum in said outer pouch is sufficient to give a pressure of from about 8 to 13 psi.

15. The device of claim 11, wherein said actuation means is a liquid and said heat generating material means is a solid adapted to react with said actuation means to cause an exothermic reaction.

16. The device of claim 15, wherein said actuation means is water and said heat generating material means is a mixture of calcium oxide and citric acid, and the weight of the heat generating material means to the volume of actuation means ranges from about 1:2 to about 1:1.

17. The device of claim 16, wherein said calcium oxide is calcined prior to admixing with said citric acid.

18. The device of claim 17, which further includes powdered zeolite admixed therein.

19. The device of claim 18, wherein the ratio of calcium oxide to citric acid to powdered zeolite is from about 14 to 20 for calcium oxide, from about 1 to 2 for citric acid, and from about 7 to 10 for powdered zeolite.

20. The device of claim 19, wherein the ratio of calcium oxide to citric acid to powdered zeolite is 17:1.5:8.5 and the ratio of the solids to the volume of water is about 3:4.
